(19) United States
(12) Patent Application Publication
(10) Pub. No.: US 2024/0033536 A1
(43) Pub. Date: Feb. 1, 2024

(54) MAT WITH DUAL LIGHT EMITTING DIODES

(71) Applicant: BioLight, Inc., Stevensville, MT (US)

(72) Inventor: Michael BELKOWSKI, Stevensville, MT (US)

(73) Assignee: Biolight, Inc, Stevensville, MT (US)

(21) Appl. No.: 18/362,533

(22) Filed: Jul. 31, 2023

Related U.S. Application Data
(60) Provisional application No. 63/369,984, filed on Aug. 1, 2022.

(51) Int. Cl. A61N 5/06; F21V 33/00

(52) U.S. Cl. CPC A61N 5/0616; F21V 33/0068; F21Y 2115/10

(57) ABSTRACT

Systems and techniques disclosed herein include a light therapy mat having a flexible clear portion, a flexible back portion, a flexible middle portion between the clear portion and the back portion, and a plurality of dual light emitting diodes (LEDs) positioned in the middle portion and configured to emit light at a first wavelength and a second wavelength. The first wavelength may be a red light wavelength or a near infrared light wavelength and the second wavelength may be the other one of the red light wavelength or the near infrared light wavelength.

### CROSS-REFERENCE TO RELATED APPLICATION

This application claims the benefit of priority to U.S. Provisional Patent Application No. 63/369,984, filed Aug. 1, 2022, the entirety of which is incorporated by reference herein.

### TECHNICAL FIELD

Various embodiments of the present disclosure relate generally to mats and, more particularly, to devices configured to provide dual light emitting diode (LED) based red and near-infrared light therapy to a user.

### INTRODUCTION

Light therapy such as sunlight exposure, vitamin D therapy, and the like have been traditionally suggested for their health benefits. However, such exposure can often be uncontrolled and may lead to conditions such as over-exposure, sunburns, and can result in increased risk of developing chronic conditions as a result of the exposure. In-home solutions provide a limited range of therapies, often with unsafe emission values and limited therapeutic benefits.

The background description provided herein is for the purpose of generally presenting the context of the disclosure. Unless otherwise indicated herein, the materials described in this section are not prior art to the claims in this application and are not admitted to be prior art, or suggestions of the prior art, by inclusion in this section.

### SUMMARY OF THE DISCLOSURE

According to an aspect of the disclosed subject matter, a light therapy mat includes a flexible clear portion; a flexible back portion; a flexible middle portion between the flexible clear portion and the flexible back portion; and a plurality of dual light emitting diodes (LEDs) positioned in the flexible middle portion and configured to emit light at a first wavelength and a second wavelength.

According to another aspect of the disclosed subject matter, a method for configuring a light therapy mat comprising a plurality of dual light emitting diodes (LEDs) includes receiving sensed data from a first sensor associated with the light therapy mat; providing the sensed data from the first sensor to a machine learning model trained by modifying one or more layers, weighs, synapses, or nodes based on historical or simulated light therapy data; receiving a first machine learning output from the machine learning model based on the sensed data from the first sensor, the first machine learning output comprising a light therapy mat configuration; and configuring the light therapy mat based on the light therapy mat configuration.

According to another aspect of the disclosed subject matter, a system for configuring a light therapy mat includes a light therapy mat comprising a plurality of dual light emitting diodes (LEDs) and a first sensor; at least one memory storing instructions; and at least one processor executing the instructions to perform a process, the processor configured to: receive sensed data from the first sensor associated with the light therapy mat; provide the sensed data from the first sensor to a machine learning model trained by modifying one or more layers, weighs, synapses, or nodes based on historical or simulated light therapy data; receive a first machine learning output from the machine learning model based on the sensed data from the first sensor, the first machine learning output comprising a light therapy mat configuration; and configure the light therapy mat based on the light therapy mat configuration.

Additional objects and advantages of the disclosed embodiments will be set forth in part in the description that follows, and in part will be apparent from the description, or may be learned by practice of the disclosed embodiments. The objects and advantages of the disclosed embodiments will be realized and attained by means of the elements and combinations particularly pointed out in the appended claims.

It is to be understood that both the foregoing general description and the following detailed description are exemplary and explanatory only and are not restrictive of the disclosed embodiments, as claimed

### BRIEF DESCRIPTION OF THE DRAWINGS

The accompanying drawings, which are incorporated in and constitute a part of this specification, illustrate various exemplary embodiments and together with the description, serve to explain the principles of the disclosed embodiments.

FIG. 1 depicts different sized mats with dual light emitting diodes, according to one or more embodiments.

FIG. 2A depicts use of a mat with dual light emitting diodes in a first position, according to one or more embodiments.

FIG. 2B depicts another use of a mat with dual light emitting diodes in a second position, according to one or more embodiments.

FIG. 2C depicts another use of a mat with dual light emitting diodes in a third position, according to one or more embodiments.

FIG. 2D depicts a flowchart for configuring a light therapy mat, according to one or more embodiments.

FIG. 3 depicts a mat with a plurality of power related accessories, according to one or more embodiments.

FIG. 4A depicts combined mats with dual light emitting diodes in a first configuration, according to one or more embodiments.

FIG. 4B depicts combined mats with dual light emitting diodes in a second configuration, according to one or more embodiments.

FIG. 5A depicts a diagram of light penetration from a red light emitted from a mat, according to one or more embodiments.

FIG. 5B depicts a diagram of light penetration from a near infrared light emitted from a mat, according to one or more embodiments.

FIG. 6 depicts a data flow for training a machine learning model, according to one or more embodiments.

FIG. 7 depicts an example system that may execute techniques presented herein.

### DETAILED DESCRIPTION OF EMBODIMENTS

The terminology used herein may be interpreted in its broadest reasonable manner, even though it is being used in conjunction with a detailed description of certain specific examples of the present disclosure. Indeed, certain terms may even be emphasized herein; however, any terminology intended to be interpreted in any restricted manner will be overtly and specifically defined as such in this Detailed Description section. Both the general description and the detailed description are exemplary and explanatory only and are not restrictive of the features, as claimed.

As used herein, the terms “comprises,” “comprising,” “having,” including,” or other variations thereof, are intended to cover a non-exclusive inclusion such that a process, method, article, or apparatus that comprises a list of elements does not include only those elements, but may include other elements not expressly listed or inherent to such a process, method, article, or apparatus.

In this disclosure, relative terms, such as, for example, “about,” “substantially,” “generally,” and “approximately” are used to indicate a possible variation of ±10% in a stated value.

The term “exemplary” is used in the sense of “example” rather than “ideal.” As used herein, the singular forms “a,” “an,” and “the” include plural reference unless the context dictates otherwise.

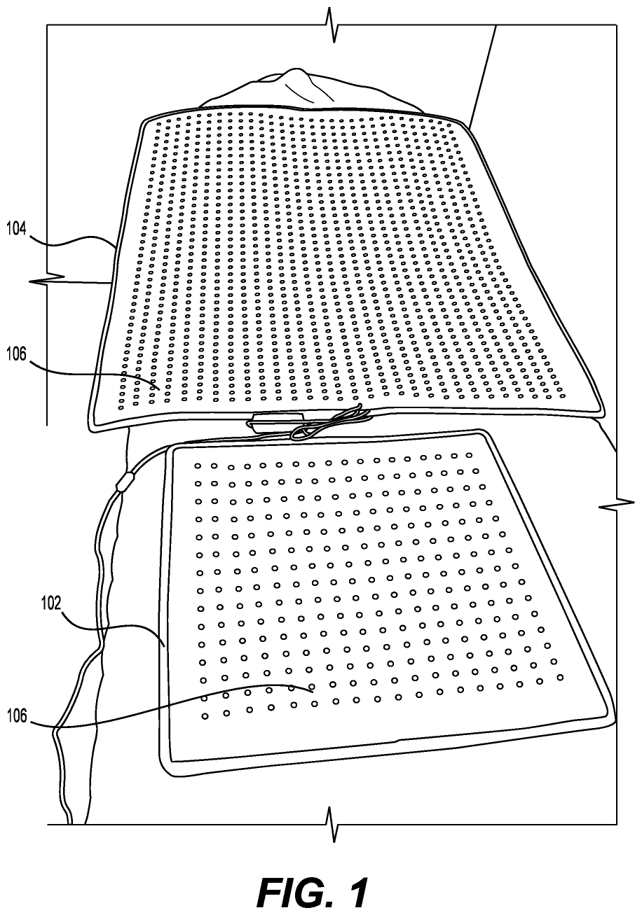

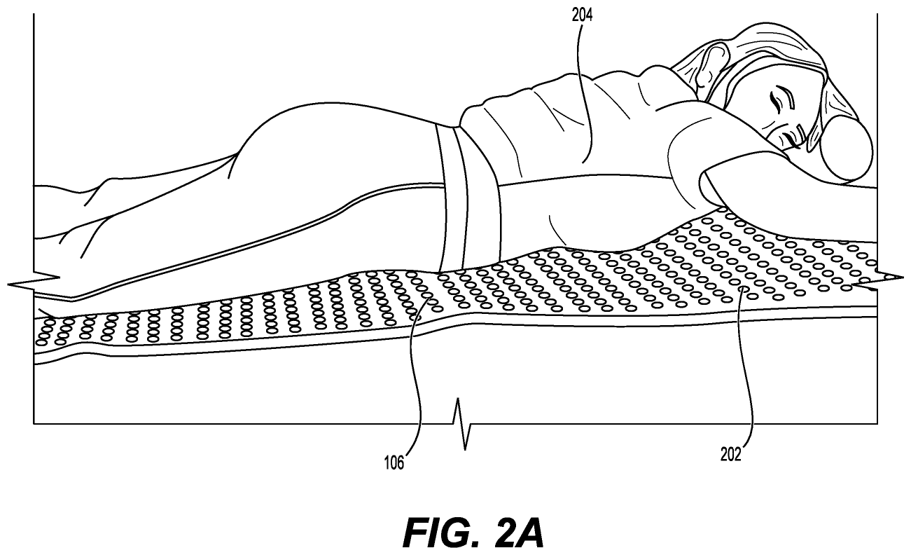

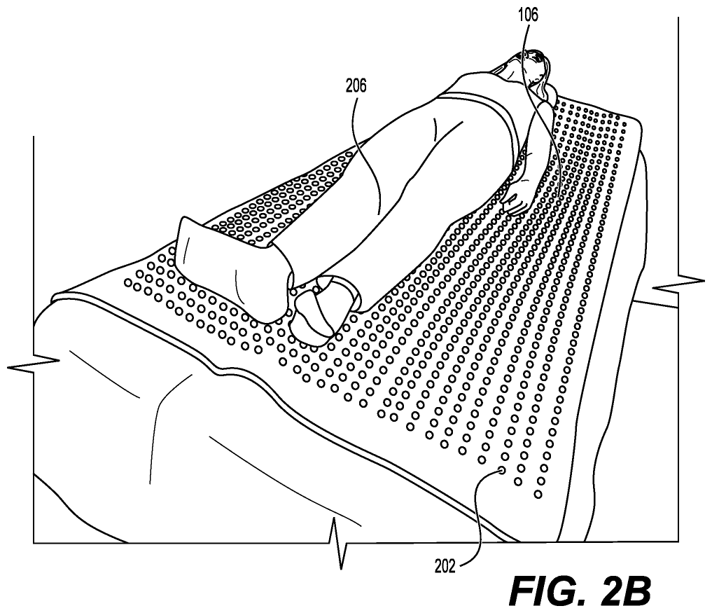

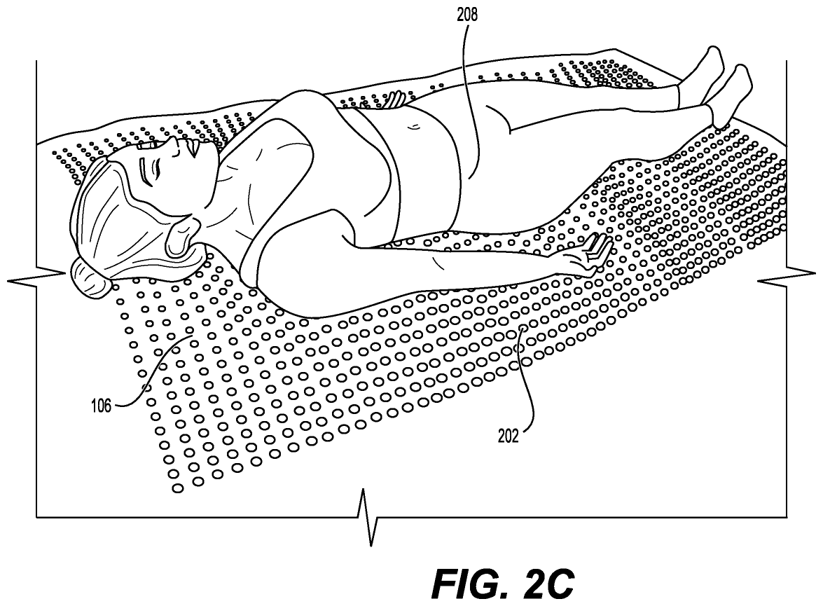

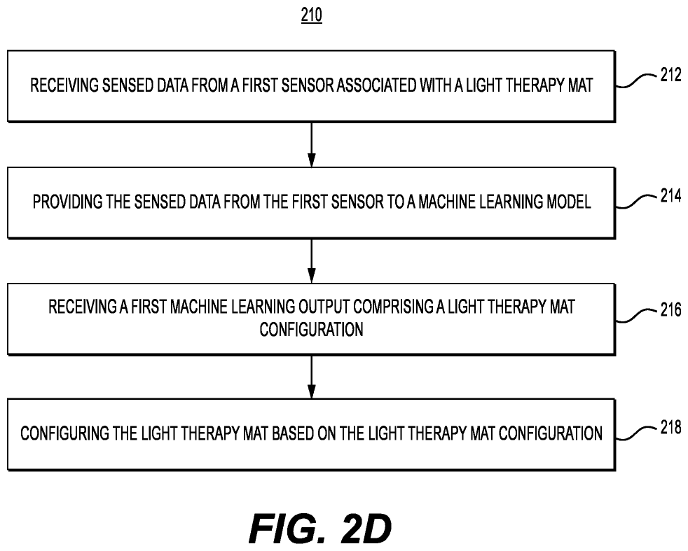

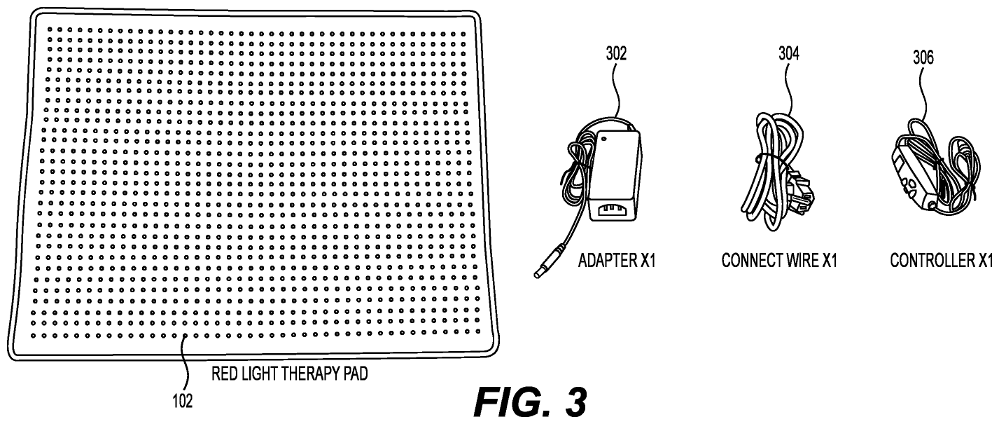

According to implementations of the disclosed subject matter, one or more light therapy mats with the dual light emitting diodes (LEDs), are provided herein. For example, these light therapy mats may include mat 102 as shown in FIGS. 1 and 3, mat 104, as shown in FIG. 1, and mat 202 as shown in FIGS. 2A, 2B, and 2C. As further disclosed herein, one difference between mat 102, mat 104, and mat 202 is the size of each respective mat. Unless specifically indicated otherwise, disclosure related to mat 102, mat 104, and/or mat 202 is provided interchangeably, such that disclosure related to one of the mats may apply to each of the other mats. As shown in FIG. 1, mat 102 may include a plurality of dual LEDs 106 that may be configured to selectively provide a red light (e.g., with a wavelength of approximately 630 nm, or within a range of approximately 600 nm-750 nm), near infrared light (e.g., with a wavelength of approximately 850 nm, or within a range of approximately 750-1000 nm), and/or a combination thereof. Generally, mat 102 may be configured to provide light in the range of approximately 600 nm to approximately 1000 nm.

Mat 102 may be configured to emit light towards a user to promote health and/or treat or mitigate health conditions such as inflammatory conditions, hair health, pain, mental health, sleep conditions, thyroid health, athletic performance, or the like. Mat 102, by emitting both the red light and the near infrared light, may reduce inflammation, increase circulation, increase fibroblast production (e.g. to allow collagen to be formed in order to build connective tissue) and optimize the functionality of mitochondria (e.g., to allow cells to generate energy more efficiently). As further discussed herein, the combination of the red light and near infrared light may be used to treat both surface conditions and sub-surface conditions (e.g. at a tissue level).

Mat 102 may be formed from any applicable material that maintains the structure of Mat 102. A clear portion of mat 102 may be flexible (e.g., not rigid) and may be composed, at least in part, of clear polyvinylchloride (PVC) material that may provide electrical insulation and/or a barrier between dual LEDs 106 of mat 102 and a user. As used herein, the term “clear” may refer to a non-opaque or partially opaque material or portion. The user may use mat 102 such that at least a portion of the user's body is in direct or indirect (e.g., through clothing) contact with the PVC material. The PVC material may include a composition that allows light emitted from dual LEDs 106 to emit through the PVC material. The light emitted from dual LEDs 106 may retain one or more light properties (e.g., wavelength, intensity, illuminance, etc.) as the light passes through the PVC material. Alternatively, or in addition, one or more properties of light emitted from dual LEDs 106 may be modified as the light passes through the PVC material. For example, a refractive index of the PVC material may modify one or more properties of the light emitted via dual LEDs 106.

At least a portion of mat 102 may be composed of a styrene butadiene rubber (SBR) composite (e.g., a middle portion between the clear portion and a back portion, as further discussed herein). The SBR composite may be coated with neoprene, polychloroprene, chloroprene rubber, and/or any applicable synthetic material which may, for example, be produced by polymerization or linking together of single molecules into larger multiple-unit molecules (e.g., of chloroprene). For example, the SBR composite may make up a portion of the mat between a clear portion (e.g., a PVC material) and a back portion, as further described herein. The SBR composite layer may be approximately 3 mm thick and may be configured to allow the mat 102 and/or dual LEDs 106 to be flexible. The dual LEDs 106 may be positioned between the clear portion and the back portion (e.g., the middle portion). The middle portion may also be flexible (e.g., not rigid). According to an implementation, the SBR composite layer may include or may be attached to a flexible printed circuit board (PCB) that connects, powers, and/or controls dual LEDs 106. According, the SBR composite layer and flexible PCB may allow mat 102 to be flexible, foldable, and/or otherwise operate or be stored in a non-rigid form. The SBR composite (e.g., neoprene) may provide high temperature resistance and protection from the heat generated by dual LEDs 106.

A back portion of mat 102 may be flexible (e.g., not rigid) and may be composed, at least in apart, of a nylon, spandex, polyurethane foam composite, and/or the like to provide flexibility and foldability for mat 102. The nylon, spandex, polyurethane foam composite, and/or the like may also be flexible such that it may allow a user to fold mat 102 and/or store mat 102 in small or tight spaces. On top of the nylon, spandex, and/or polyurethane foam composite (e.g., between the back portion and the clear portion), mat 102 may include a thin memory foam layer that provides padding, resulting in a more comfortable experience for a user while using mat 102. The memory foam may be approximately 0.5″-1″ thick and configured to mold to a user's body, in response to pressure from lying on mat 102, which may result in an approximately even distribution of body weight.

According to an implementation, non-skid material (e.g., a non-skid SBR) may be included on a back side (e.g., a side opposite of the clear portion) of mat 102 for slip resistance on surfaces. Mat 102 may also include a top layer (e.g., a polyurethane layer) that absorbs or deflect a user's sweat (e.g., generated from the heat generated by dual LEDs 106 and/or by user excursion) and may further prevent or mitigate sliding during use of mat 102. The top layer (e.g., polyurethane layer) may also be configured to absorb or deflect other fluids such water.

According to an implementation, at least a portion of mat 102 may be composed of a polystyrene sticky material which adheres to and/or increases friction with one or more surfaces (e.g., using a web coating without any residue). For example, all or a portion of the back side of mat 102 may include the polystyrene sticky material. As another example, mat 102 may be framed (e.g., bound) by the polystyrene sticky material. The polystyrene sticky material may allow for easy relocation of mat 102 and may be configured to be removed (e.g., to perform maintenance on mat 102 or to clean portions of mat 102). The polystyrene sticky material may be constructed with a polystyrene material with an approximately ⅛″ thick poly-vinyl web coating on the back side (e.g., underside) of mat 102, for increased traction (e.g., friction) with a surface.

According to implementations of the disclosed matter, a portion of mat 102 (e.g., an outside edge of mat 102) may have magnetic components (e.g., strips) with adhesive. The magnetic components may allow a user to hang mat 102 on surfaces (e.g., walls) without damaging or chipping the surfaces. Mat 102 may also include one or more fasteners or clasps such as a clip attachment (e.g., a double sided clip attachment) to attach mat 102, for example, on a hook. The one or more fasteners or claps may be configured to hang mat 102 (e.g., on a wall, a hook, etc.) and/or to clip mat 102 to objects (e.g., a computer screen, a hanging rod, etc.).

Mat 102 may include or otherwise be associated with one or more electrical components such as a memory, a processor, a controller, a timer, an electronic display, etc. For example, such electrical components may be housed within mat 102 or may be attached to mat 102.

Mat 102 may include one or more input receptors which may be knobs, buttons, touch points (e.g., haptic response points), electronic signal receiver (e.g., via a controller) or the like, that may be accessible to a user or a machine learning output. An input receptor including an electronic signal receiver may configure or otherwise operate one or more other input receptors (e.g., power button) or other electronic components (e.g., timer, dual LEDs 106, etc.) disclosed here. Alternatively or in addition, the input receptors may be or may include input sensors for voice and/or gesture activation. The input receptors may include a power button to configure mat 102 between an on or off state. According to implementations, mat 102 may include a timer, a setting adjustor, or the like. According to an implementation, two or more tasks may be performed by the same input receptor (e.g., power and timer operation may be conducted using the same input receptor). The input receptors may be used to adjust a configuration of mat 102 which may include a wavelength, an intensity, a duration of activation, or the like. A light therapy mat (e.g., mat 102) configuration is generally referred to as a “setting” herein. The settings may be categorized by health conditions (e.g., a hair treatment setting, a muscle treatment setting, etc.), such that a user may select a health condition and mat 102 may be configured based on the selection. The settings may configure mat 102 by adjusting one or more properties such as one or more of a wavelength, intensity (e.g. may approximately be a low setting at 1 and the highest setting at 5), duration, or the like based on the setting itself (e.g., if the setting adjusts one or more of the wavelength, intensity, duration) or based on the selection of a treatment modality (e.g., based on a health condition). As an example, mat 102 may be configured using a plurality of intensity settings (e.g., up to 5 discrete settings, up to 10 discrete settings, a gradient of settings from a lowest to a highest intensity, etc.). The intensity may be adjusted by user input (e.g., via one or more input receptors, as discussed herein) and/or automatically based on an output (e.g., based on a machine learning output, as discussed herein).

Mat 102 may include a timer or a timer function configured to automatically shut-off mat 102 after a given amount of time. The given amount of time may be pre-determined or dynamically determined. The timer may be configured based on a time that the user may select, for example, the time may range from approximately from 5 minutes to approximately 30 minutes (e.g., approximately 5 minutes, approximately 10 minutes, approximately 15 minutes, approximately 20 minutes, approximately 25 minutes, approximately 30 minutes, etc.). A pre-determined amount of time may be set during manufacture of mat 102 (e.g., may be approximately 8 minutes, approximately 4 minutes, etc.) and/or may be determined based on the intensity of the light (e.g., red light, infrared light) being emitted. For example, a user may set the timer while using mat 102 at a higher light intensity setting. The timer may automatically be set for four minutes based on the higher light intensity setting. At a different time, the user may set the timer while using mat 102 at a lower light intensity setting relative to the higher intensity setting. The timer may automatically be set for eight minutes based on the lower light intensity setting and may be controlled by an input receptor or an external control (e.g., an application with a GUI that receives input from a user).

According to an implementation of the disclosed subject matter, mat 102 may include or be operably coupled to a sensor to detect a health condition and/or an effect of a health condition on a user. The health condition may be any applicable condition including, but not limited to, inflammatory conditions, hair health, muscle condition, stress condition, sleep conditions, thyroid health, activity performance, or the like. The sensor may be a fitness tracker, visual sensor, ambient condition detection sensor, pH sensor, biochemical sensor, or the like. A sensor may be attached to a portion of the mat or may be configured to communicate with mat 102. For example, a sensor may be part of a wearable device that tracks a user's sleep and/or activity. The tracking may be conducted using signals such as movement signals, heart rate signals, respiration rate signals, temperature signals (e.g., skin temperature signals), blood oxygen saturation signals, heart rate variability signals, heart rate reserve signals, and/or the like, which may be sensed and/or generated using applicable sensors. The wearable device may communicate with mat 102 in a wired or wireless manner or may communicate with a controller (e.g., a mobile device) that is in communication with mat 102. Mat 102 may be configured to adjust a setting (e.g., a light property) based on sensed data (e.g., data sensed by a sensor, data determined based on signals sensed by a sensor, converted signals, etc.). For example, the sensor may sense data about a user, an ambient condition, and/or a mat property, and provide the sensed data to a machine learning model. Such sensed data may include one or more of movement signals, heart rate signals, respiration rate signals, temperature signals (e.g., skin temperature signals), blood oxygen saturation signals, heart rate variability signals, heart rate reserve signals, and/or the like. The machine learning model may be trained to output one or more settings of mat 102 based on the input sensor data (e.g., one or more settings discussed herein). The output may be based on detecting a given health condition, user biometrics, user condition, user activity, and/or the like. The output may be based on detecting sensed properties (e.g., sensed data) that are used to determine a given output (e.g., a light property or mat configuration). The given output may be further based on historical data (e.g., for a user, or a cohort of users), or the like. The machine learning model may be trained by adjusting one or more weights, layers, synapses, nodes, etc., based on historical, cohort, and/or simulated light therapy data (e.g., data indicating how components of a light therapy mat are configured in response to sensed data, data indicating configurations of a light therapy mat that result in a desired result, etc.). For example, the sensor data may indicate a given health condition (e.g., as determined by the a machine learning model or algorithm) and the machine learning model may output one or more parameters to operate mat 102 in order to treat and/or mitigate the health condition. Alternatively, the machine learning model may be used as a clinical decision support engine that may receive information from clinical guidelines such as a user's doctor, coach, physical therapist, care taker, or the like, (e.g., via a server, network, or other connection to the user's data). Based on the user's health information, the machine learning model may output parameters for operation of mat 102.

The parameters may cause mat 102 to, for example, output optimal light therapy to treat or mitigate any conditions included in the health information. Alternatively, or in addition, the machine learning model may receive inputs from the user. For example, the user may indicate acne as a health condition and the input may be provided to the machine learning model to provide outputs to optimize acne treatment. Alternatively, machine learning model may output parameters for operation of mat 102 based on a user's health goals, prior treatment history, and biological makeup (e.g. gender, weight, height, etc.). The output may include a wavelength or set of wavelengths to output using mat 102. The output may also or alternatively include a duration data such that the timer can be dynamically set to turn mat 102 off after the duration of time output by the machine learning model. Alternatively, the timer may be dynamically set to change a wavelength after a duration of time output by the machine learning model.

According to an implementation of the disclosed subject matter, setting for mat 102 may be adjusted based on user or automated input. As disclosed above, a machine learning model may output settings adjustments (e.g., intensity, wavelength, duration, etc.) based on sensor data and/or data received at the machine learning model. One or more setting of mat 102 may be adjusted on user input. A user may provide the user input directly via input recipients on mat 102. Alternatively, mat 102 may be connected to a user device (e.g., via a network, wired, or other wireless connection). The user may connect to mat 102 via an application (e.g. a mobile device application, website or web application, etc.) or a standalone controller 306 and provide setting input via a graphical user interface (GUI) of the application.

According to an implementation of the disclosed subject matter, mat 102 may include one or more pressure sensors that may communicate with part of mat 102 (e.g., a processor) or with an external component (e.g., an external processor, a mobile device, etc.) in communication with mat 102. The pressure sensors may be configured to calculate pressure applied (e.g., amount of pressure, change in pressure, regions where pressure is applied), weight, and/or a weight distribution of the user. The pressure and/or weight information may be input into a machine learning model, as disclosed above, where it may output setting adjustments (e.g., intensity, wavelength, duration, etc.) based on the pressure sensor data. Pressure sensor data and/or machine learning outputs may be sent through a network or wireless connection to a user's device to generate light property outputs (e.g., light intensity, duration, wavelength, etc.) based on use of mat 102 by a given user. The one or more pressure sensors may be configured to detect one or more regions of mat 102 that are in contact with a user such that, for example, only dual LEDs 106 in that region are activated, while deactivating other dual LEDs 106 to conserve energy and dual LEDs 106 lifespan. The pressure sensors may detect movement of the user and, based on the movement and corresponding changes in pressure, may adjust which dual LEDs 106 are activated and which dual LEDs are deactivated at a given time.

For example, a user may be positioned at a first area of mat 102, and the first area may be detected by one or more pressure sensors. One or more biometric sensors may detect user biometric data and may provide the user biometric data to a machine learning model. The machine learning model may output mat 102 settings based on the pressure sensors and/or biometric sensors. As a result of the machine learning model output, a subset of mat 102 dual LEDs 106 may be activated (e.g., those corresponding to the first area of mat 102 that the user is positioned on). The subset of mat 102 dual LEDs 106 may be configured to output one or more wavelengths (e.g., alternating between two or more wavelengths every 30 seconds) of light for one or more given durations (e.g., 4 minutes on and 5 minutes off in a cyclical manner for 2 hours) and/or at given intensities (e.g., at 40% intensity for the first 4 minutes and at 60% intensity for the second 5 minutes). If the user changes her position during a given treatment period, the subset of dual LEDs 106 may change from a first set to a second set, based on updated pressure sensor data. It will be understood that mat 102 settings may be different for different users based on each user's respective pressure sensor data and/or biometric sensor data.

According to an implementation of the disclosed subject matter, mat 102 may include a temperature sensor that may communicate with part of mat 102 (e.g., a processor) or with an external component (e.g., an external processor, a mobile device, etc.) in communication with mat 102. The temperature sensor may be configured to detect the temperature of different regions of a user's body (e.g., as a user uses mat 102). The temperature data may be input into a machine learning model, as disclosed above, where the machine learning model may output adjustments (e.g., intensity, wavelength, duration, etc.) based on temperature sensor data. The temperature sensor data may be used to modify light properties (e.g., increase or decrease the intensity of light) for certain regions of the mat. For example, if the user is laying on top of mat 102 and the temperature is above a threshold for a particular region of the user's body, the temperature sensor data may cause a decrease in the intensity of light at that region, while keeping the intensity the same in other parts of mat 102. Furthermore, the one or more sensors (e.g., temperature sensors, optical sensors, etc.) may be configured to detect blood flow. Based on the detected blood flow, on more light properties of light emitted by dual LEDs 106 may be adjusted in a manner similar to that described herein in relation to pressure sensors and/or temperature sensors.

According to an implementation of the disclosed subject matter, mat 102 may include a moisture sensor. The moisture sensor may be configured to detect how much sweat a user generates and/or an amount of fluid (e.g., water) the user is losing while using mat 102. The data may be inputted into a machine learning model, as disclosed above, which may output adjusted light properties (e.g., intensity, wavelength, duration, etc.) based on the sweat and/or fluid loss data.

According to an implementation of the disclosed subject matter, mat 102 may include a piezoelectric sensor. The piezoelectric sensor may detect pressure differences in the air resulting from the users breathing. The data may be inputted into a machine learning model, as disclosed above, which may output adjusted light properties (e.g., intensity, wavelength, duration, etc.) based on the sweat and/or fluid loss data.

According to an implementation of the disclosed subject matter, mat 102 may include a visual sensor (e.g., a camera) configured to scan all or part of a user's body. Such scanned data may be input into a machine learning model, as disclosed above, which may output adjusted light properties (e.g., intensity, wavelength, duration, etc.) based on the sweat and/or fluid loss data. (e.g., for a particular region of the user's body). Additionally, the scanned data may be used to predict potential health conditions and/or to determine therapies (e.g., light therapy).

Properties of mat 102 may be adjusted based on prior use by a given user. The prior use may be associated with a given user's user profile which may be stored local to mat 102 or may be stored at a remote storage (e.g., server, database, etc.) in communication with mat 102. A given user's historical use of mat 102 may be used to determine mat 102 settings. For example, a machine learning model may generate output mat 102 settings based at least in part on a given user's historical use of mat 102 (e.g., based on the user's profile). Moreover, if multiple users use the same mat 102, visual sensor may identify a user's face and machine learning model may automatically adjust the settings of mat 102 based on prior sensor and treatment data.

According to an implementation of the disclosed subject matter, mat 102 may include a microphone configured to recognize a user's voice. For example, if multiple users use the same mat 102, a microphone may identify a user's voice and machine learning model may automatically adjust the settings of mat 102 based on prior sensor and treatment data, as discussed herein.

Accordingly, mat 102 may include one or more sensors or may be associated with one or more sensors that detect sensed data (e.g., temperature data, pressure data, blood flow data, moisture data, visual data, etc.) which may be transmitted to a processor or an external device such as a user's device (e.g., mobile phone, watch, etc.). The sensed data may be used to determine optimal light properties to improve a user condition (e.g., a health condition). For example, the sensed data may be processed by a machine learning model, as disclosed above, to determine optimal light properties to be output using mat 102. According to an implementation, the sensed data and/or a condition or treatment identified based on the sensed data may be transmitted to an external system such as to a health care provider.

According to an implementation, sensed data (e.g., pressure data, breath data, etc.) may be used to adjust light properties such that the adjustment includes a fluctuation of emitted wavelengths and/or intensity. The fluctuation may mimic the cadence of a user's breathing pattern. For example, if the user is breathing rapidly, dual LEDs 106 may fluctuate intensity a more rapid pace compared to if the user is breathing at a slower pace during use of mat 102. The fluctuation of light properties may mimic a breathing cadence such that, for example, intensity increases as a user breathes in, and decreases as a user breathes out. Alternatively, for example, intensity may increase as the user breathes out and decrease as the user breathes in. According to an implementation, light properties may fluctuate based on a target cadence (e.g., as identified by the output of a machine learning model) to encourage a user to match the target cadence. The target cadence may be output based on, for example, a user condition or a target user state. For example, a sensor (e.g., pressure sensor, breath sensor, motion sensor, etc.) output may be used to detect a user's breathing cadence that is too fast for a resting state. Accordingly, a machine learning model may output the target cadence, based on the user's breathing cadence, user health history, a resting cadence, and/or the like. According to an implementation, the fluctuation may be gradual such that increases and/or decreases in light properties are based on a gradient.

As shown in FIGS. 1, 2A, 2B, and 2C, mat 102, mat 104, and/or mat 202 may include a plurality of dual LEDs 106 integrated between the clear portion and the back portion of the respective mat. The dual LEDs 106 may be manufactured or placed on strips that are inserted or placed on a flexible PCB, as disclosed herein. Alternatively or in addition, the dual LEDs 106 may be manufactured as a sheet that is applied to mat 102. Alternatively, the dual LEDs 106 may be individually placed in mat 102. The dual LEDs may be equidistant from each other or may be spaced such that the outside edges have a higher distribution of dual LEDs 106 and a central part has a lower distribution of dual LEDs 106, or vice versa. Alternatively, the dual LEDs 106 may be arranged in any other pattern such as a circular pattern, random pattern, a design, or the like). The rows may be staggered such that the electronics for the dual LEDs 106 have sufficient space to separate from each other.

According to implementations of the disclosed subject matter, mat 102 may incorporate security features. For example, mat 102 may include a time used detection mechanism to detect how long a user has used mat 102 and/or at what intensity mat 102 was used for the duration. If the time used detection mechanism determines that mat 102 was used for a duration greater than a recommended duration and/or at an intensity greater than a recommended intensity for a given duration, the time used detection mechanism may automatically shut off mat 102 or reduce mat 102.

The irradiance of mat 102, 104, and mat 202 may be approximately greater than 100 mW/cm2. For example, the irradiance of mat 102 may be approximately 150 mW/cm2. The irradiance may be variable based on one or more settings (e.g., as output by a machine learning model, set by a user, etc.).

FIG. 1 shows an example of two different size mats, mat 102 and mat 104, with dual LEDs. As shown, mat 102 may be smaller than mat 104. Mat 104 may be approximately 38″ in length and approximately 30″ in width compared to mat 102 which may approximately be 19″ in length and approximately 15″ in width. Additionally, mat 104 may use approximately 100 W of power compared to mat 102 which may use approximately 40 W of power. Mat 104 may include, for example, approximately 40 rows of 31 dual LEDs each (approximately 1230 dual LEDs). Mat 102 may include, for example, 30 rows of 9 dual LEDs each (approximately 270 dual LEDs). It will be understood that mat 102 and/or 104 may be of variable lengths, widths, power, and number of dual LEDs other than those disclosed herein.

As disclosed herein in reference to mat 102, mat 104 may be configured to provide red light (e.g., with a wavelength of approximately 630 nm, or a range of approximately 600 nm-750 nm), near infrared light (e.g., with a wavelength of approximately 850 nm, or a range of approximately 750-1000 nm), or a combination thereof. Generally, Mat 104 may be configured to provide light in the range of approximately 600 nm-approximately 1000 nm. According to an implementation, when emitting either red light or near infrared light, each of the dual LEDs may emit the respective frequency of lights (i.e., not a subset of the dual LEDs).

FIGS. 2A, 2B, and 2C depict examples of a full body mat 202. It will be understood that the disclosure provided herein for mat 102 and mat 104 generally applies to mat 202. Mat 202 may be approximately 71″ in length and approximately 35″ in width and may use approximately 200 W of power. Mat 202 may include, for example, approximately 40 rows of approximately 52 dual LEDs each (approximately 2130 dual LEDs). It will be understood that mat 202 may be of variable lengths, widths, power, and number of dual LEDs other than those disclosed herein. According to an implementation, mat 202 may include one or more repeaters to transmit power (e.g., from a battery or power source) across the entire mat 202.

As disclosed herein in reference to mat 102 and mat 104, mat 202 may be configured to provide red light (e.g., with a wavelength of approximately 630 nm, or a range of approximately 600 nm-750 nm), near infrared light (e.g., with a wavelength of approximately 850 nm, or a range of approximately 750-1000 nm), or a combination thereof. Generally, mat 202 may be configured to provide light in the range of approximately 600 nm-approximately 1000 nm. According to an implementation, when emitting either red light or near infrared light, each of the dual LEDs emit the respective frequency of lights (i.e., not a subset of the dual LEDs).

FIG. 2A depicts use of mat 202 in a first user position 204. FIG. 2B depicts use of mat 202 in a second user position 206. FIG. 2C depicts use of mat 202 in a third user position 208. It will be understood that the mats disclosed herein may be used in any applicable position or orientation relative to a user's body. For example, if the user wants to target light emitted by dual LEDs 106 on a particular region of the body, the user may lay or position herself in a position that exposes the respective particular region of the body to the dual LEDs 106 (e.g., a user is not limited to a given position such as an upright position when using the mats disclosed herein). The user may input a targeted body portion or a health condition (e.g., via an input receptor, a mobile device in communication with a mat, etc.). Mat 202 may configure one or more dual LEDs 106 based on one or more light properties and/or one or more optimal positions for a user, based on the user input. As disclosed herein, one or more dual LEDs 106 may be configured using a machine learning output generated at least in part based on the user input. Additionally, while a user is in a particular position (e.g., position 204, 206, 208, etc.), one or more sensors (e.g., pressure sensors) associated with mat 202 may output sensed data indicative of the one or more positions. Light properties, including whether one or more dual LEDs 106 is activated or deactivated may be determined based on the sensor data (e.g., by a machine learning output).

According to an implementation, a mat disclosed herein may be placed in a gym or a workout class setting. One or more light properties of the respective mat may be configured based on user activity at the gym or workout class setting. One or more sensors associated with a mat (e.g., mat 202) may sense data associated with the user during a workout, a warmup, a cool-down, or the like. Light properties of dual LEDs 106 of the mat may configured based on the sensed data and may update continuously or periodically to adjust for changes in sensed data (e.g., changes in user heartbeat, sweat amount, excursion, biometric data, motion data, etc.). For example, a user may perform a yoga workout while laying on mat 202. During the yoga workout, dual LEDs 106 may activate, deactivate, or modify other light properties based on pressure sensors detecting positions of the user, based on motion sensors, based on sweat sensors, or the like, as discussed herein. When a user exerts pressure on specific parts of mat 202 (e.g., when only the hands and feet of the user are in contact with the mat), dual LEDs 106 positioned in the areas of mat 202 that receive the pressure may emit light at a high intensity while other dual LEDs of mat 202 may emit light at a low intensity, relative to the high intensity. Similarly, one or more mats disclosed herein may be integrated into sauna rooms, massage therapy (e.g., rooms, tables, etc.), locker rooms, meditation spaces, or the like. For example, one or more mats disclose herein may be attached or integrated with a massage therapy table such that the one or more mats remain affixed to the massage therapy table even as a user using the table moves.

According to implementations of the disclosed subject matter, one or more mats disclosed herein may be attached to or integrated with travel components. For example, one or more mats disclosed herein may be integrated into a vehicle component (e.g., a car seat base, a car seat back, a trunk bed, a train bed, an airplane seat base, an airplane seat back, a motorcycle seat, a camper bed, etc.). A mat integrated into a vehicle component may be powered by a vehicle battery, engine, and/or any other mechanism used to power one or more vehicle components.

According to implementations of the disclosed subject matter, one or more mats disclosed herein may be used in a living space (e.g., a house, a hotel, a dorm, an apartment, etc.). The one or more mats may be placed on a ground (e.g., where a user may exercise, meditate, etc.), or may be placed on a surface at an angle relative to a ground such as on a vertical surface (e.g., as a drape, a shower curtain, a wall hanging) and/or any other applicable surface. The one or more mats may be placed around a user's work station (e.g., around a desktop or laptop area such as under a user's forearms as the user types, on an office chair base or back, etc.).

According to an implementation, mat 102 may be integrated with a speaker to provide sound therapy (e.g., integrated sound therapy). A speaker or one or more speakers may be part of mat 102 and/or may be in communication with a component of mat 102. The output of a health sensor that tracks user attributes (e.g., brain activity) may be provided to a machine learning model that outputs a signal that causes a change in an audio property emitted by a speaker integrated into mat 102 or in communication with a component of mat 102. For example, one or more frequencies (e.g., sound emitted at a given Hz value) may contribute to improving brain function in users (e.g., in Alzheimer's patients). Accordingly, sound therapy based on health sensor data may be provided in combination with light therapy, as disclosed herein. Further, the combination of sound and light therapy, as disclosed herein, may reduce amyloid and Tau pathology in the sensory cortex, in the hippocampus, or other applicable part of the human body. For example, dual LEDs 106 of mat 102 may be configured to flicker at a given frequency (e.g., approximately 40 Hz) during use by a user, for a given amount of time (e.g., approximately one hour), for a given number of days. At the same time or at a different time, the user may be exposed to sound at a similar frequency (e.g., approximately 40 Hz), for a given amount of time (e.g., approximately one hour), for a given number of days. The light and/or sound exposure may reduce, for example, beta-amyloid levels in the auditory cortex, the hippocampus, a different brain region for processing and/or recalling memories, or the like. The light and sound exposure may reduce or eliminate beta-amyloid plaques in additional brain regions such as the prefrontal cortex and may result in a boost to microglial activity.

According to an implementation, mat 102 may be integrated with a copper coil to provide pulsed electromagnetic field (PEMF) therapy. A copper coil or one or more copper coils may be part of mat 102 and/or may be in communication with a component of mat 102. A copper coil or one or more copper coils may be coupled with a frequency generator to produce an electrical signal to create a pulsed electromagnetic field. The output of a health sensor that tracks user attributes (e.g., inflammation) may be provided to a machine leaning model that outputs a signal that causes a change in an electromagnetic property emitted by a copper coil integrated into mat 102 or in communication with a component of mat 102. For example, one or more frequencies (e.g. electromagnetic waves emitted at a given Hz value) may contribute to improving chronic inflammation in joints or the soft tissue. Accordingly, PEMF therapy based on health sensor data may be provided in combination with light therapy, as disclosed herein. Further, the combination of PEMF therapy and light therapy, as disclosed herein, may reduce inflammation and pain, increase electrolytes and ions to increase cellular metabolism, and promote the synthesis of skeletal extracellular matrix to facilitate healing. For example, dual LEDs 106 of mat 102 may be configured to flicker at a given frequency (e.g., approximately 20 Hz) during use by a user, for a given amount of time (e.g., approximately 20 minutes), for a given number of days. At the same time or at a different time, the user may be exposed to electromagnetic waves at a similar frequency (e.g., approximately 20 Hz), for a given amount of time (e.g., approximately 20 minutes), for a given number of days.

According to an implementation, mat 102 may be integrated with an electrode pad to provide micro current electrical neuromuscular stimulation (MENS) therapy. An electrode pad or one or more electrode pads may be part of mat 102 and/or may be in communication with a component of mat 102. The output of a health sensor that tracks user attributes (e.g., inflammation) may be provided to a machine leaning model that outputs a signal that causes a change in electrical property outputted by electrode pad integrated into mat 102 or in communication with a component of mat 102. For example, one or more frequencies (e.g. electric stimulation in Hz value) combined with one or more currents contribute to healing skeletal muscular tissue. Accordingly, MENS therapy based on health sensor data may be provided in combination with light therapy, as disclosed herein. Further, the combination of MENS therapy and light therapy, as disclosed herein, may reduce inflammation and pain, increase ATP production, and loosen or soften tissues of muscle to relieve pain. For example, dual LEDs 106 of mat 102 may be configured to flicker at a given frequency (e.g., approximately 10 Hz) during use by a user, for a given amount of time (e.g., approximately 5 minutes), for a given number of days. At the same time or at a different time, the user may be exposed to current at a similar frequency (e.g., approximately 10 Hz), for a given amount of time (e.g., approximately 5 minutes), for a given number of days.

According to an implementation, mat 102 may be integrated with a motor to provide vibration therapy. A motor or one or more motors may be part of mat 102 and/or may be in communication with a component of mat 102. The output of a health sensor that tracks user attributes (e.g., inflammation) may be provided to a machine leaning model that outputs a signal that causes a change in vibrational property outputted by a motor integrated into mat 102 or in communication with a component of mat 102. For example, one or more frequencies (e.g. vibration in Hz value) combined with one or more vibrational intensities contribute to improving blood circulation. Accordingly, vibrational therapy based on health sensor data may be provided in combination with light therapy, as disclosed herein. Further, the combination of MENS therapy and light therapy, as disclosed herein, may reduce inflammation and pain, may contract and relax muscle tissue to reduce muscle soreness, may increase osteoblasts to increase bone density. For example, dual LEDs 106 of mat 102 may be configured to flicker at a given frequency (e.g., approximately 30 Hz) during use by a user, for a given amount of time (e.g., approximately 20 minutes), for a given number of days. At the same time or at a different time, the user may be exposed to vibration at a similar frequency (e.g., approximately 30 Hz), for a given amount of time (e.g., approximately 20 minutes), for a given number of days.

FIG. 2D shows a flowchart 210 for configuring a light therapy mat (e.g., mat 102, mat 104, mat 202, etc.), in accordance with the techniques disclosed herein. At step 212 of flowchart 210, sensed data from one or more sensors associated with a light therapy mat may be received. For example, the sensed data may be received at an electronic component associated with the light therapy mat (e.g., a memory, a processor, etc.).

At step 214, the sensed data from the one or more sensors may be provided as an input to a machine learning model, as disclosed herein. For example, the sensed data may be provided as an input to the machine learning model via a processor. The machine learning model may be stored at or accessed via one or more electrical components associated with the light therapy mat. For example, the machine learning model may be implemented using one or more remote (e.g., cloud) components which may be accessed via a processor associated with the light therapy mat.

At step 216, a machine learning output including a light therapy mat configuration may be received. The light therapy mat configuration may be any light therapy mat setting (e.g., a setting of a component of and/or associated with a light therapy mat). For example, the light therapy mat configuration may include one or more settings or properties such as wavelengths of light, intensities of light, rates of activation, durations of activation, or frequencies associated with the light therapy mat. The light therapy mat configuration may cause one or more components (e.g., dual LEDs 106) to activate in accordance with such settings or properties and may further include when a given set of settings or properties are activated. For example, the light therapy mat configuration may cycle through two sets of settings. A first setting may include a first wavelength, a first intensity, and/or may be activated for a first duration of time. A second setting may include a second wavelength, a second intensity, and/or may be activated for a second duration of time. It will be understood that although the example above includes cycling through two sets of settings, a given set of settings may be iterated once or any number of times (e.g., may not be cyclical), may include one or more other settings or properties, and the like. At step 218, the light therapy mat may be configured in accordance with the light therapy configuration output by the machine learning model.

As shown in FIG. 3, mat 102 may be powered via a direct power connection provided via adaptor 302 and/or connection wire 304. Alternatively, or in addition, mat 102 may be powered via a battery (not shown). A battery may be a power bank (e.g., configured to power an approximately 40 W, approximately 100 W, and/or approximately 200 W mat) charged in any applicable manner such as a Universal Serial Bus (USB) charger, wireless (e.g., Qi) charger, magnetic connection charger, adaptor 302, and/or connection wire 304, and/or the like. Mat 102 may include an indication of a low battery directly on mat 102 and/or may provide the indication via a user device (e.g., mobile phone) in connection with mat 102. Accordingly, mat 102 may be powered by a power connection, may be powered by a batter, and/or both. For example, a user may charge a battery for mat 102 using the user's vehicle during a camping trip and may use mat 102 in a tent, away from the vehicle's power source.

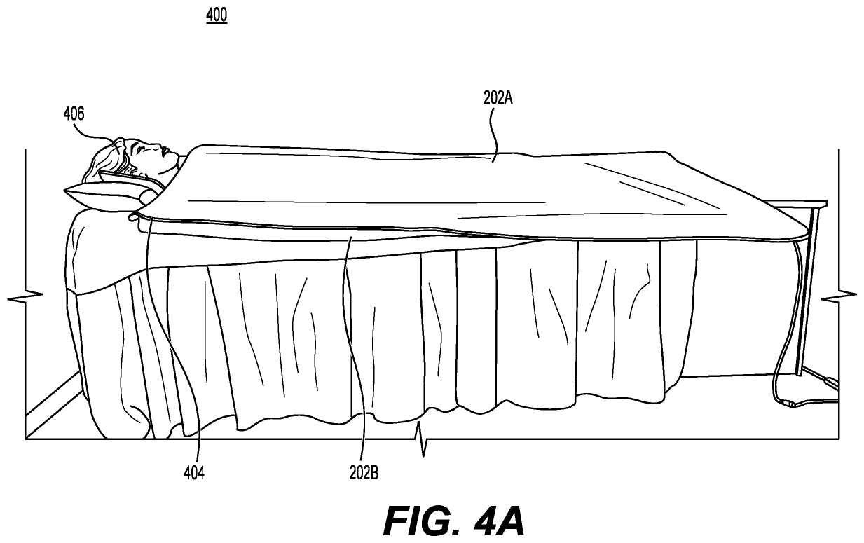

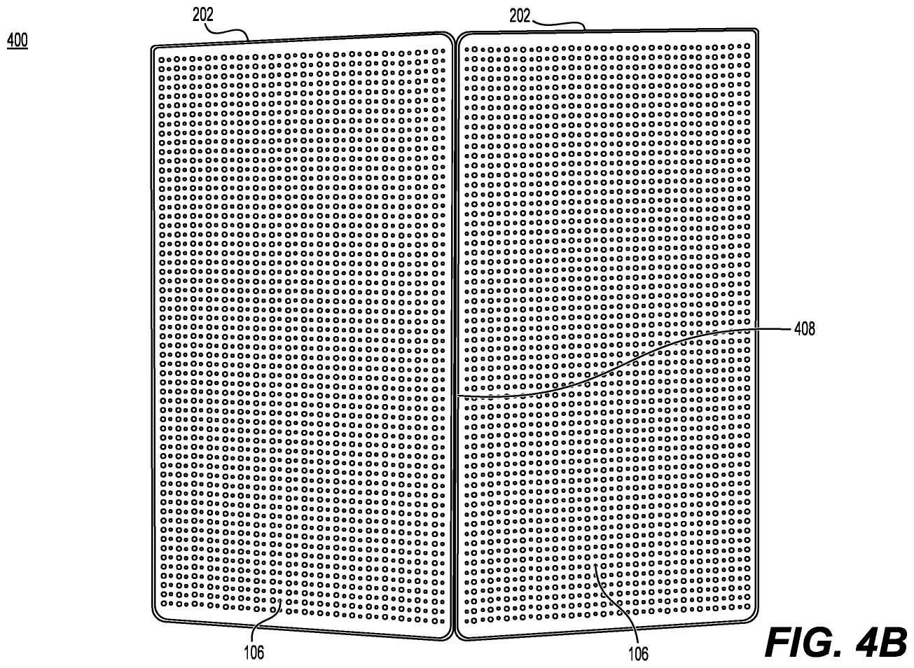

FIG. 4A depicts combined mats 400 with dual light emitting diodes in a first configuration. As shown in FIG. 4A, a first mat 202A may be combined with a second mat 202B to generate combined mats 400. Dual LEDs 106 of first mat 202A may face the light emitting diodes of second mat 202B when in the first configuration. At an outside edge of first mat 202A and second mat 202B, first mat 202A and second mat 202B may include a connector (e.g., zipper 404) to connect the first mat 202A and second mat 202B and/or to enclose user 406 between first mat 202A and second mat 202B. On an opposite outside edge from the zipper 404 of first mat 202A and second mat 202B, first mat 202A and second mat 202 may be combined together with a material 408 (e.g., an adhesive material, glue, Velcro®, etc.) as shown in FIG. 4B. The combination of first mat 202A and second mat 202B may allow for the user to experience red light therapy on different portions (e.g., opposite sides) of the user's body. Light emitted by dual LEDs 106 by first mat 202A and second mat 202B may be targeted on different (e.g., opposite) regions of a user's body. For example, the user may lay or position him or herself in a position that exposes the respective particular regions of the body to the dual LEDs 106 of first mat 202A and second mat 202B. Alternatively, a continuous mat having dimensions that extend around a user as shown via mat 202A and 202B may be used in accordance with the techniques disclosed herein.

According to an implementation of the disclosed subject matter, settings for first mat 202A may be different from the settings for second mat 202B based on user input or automated input. For example, for treatment focused on a particular region of the body facing first mat 202A, the user or machine learning output may cause an increase or decrease the wavelength, intensity, duration of activation of the dual LEDs 106 of first mat 202A separate from second mat 202B. Alternatively, as disclosed above, a machine learning model may output settings adjustments for first mat 202A and second mat 202B (e.g., intensity, wavelength, duration, etc.) based on sensor data and/or data received at the machine learning model.

Accordingly, for example, a first subset of dual LEDs 106 of first mat 202A may be configured to output light having settings different than a second subset of dual LEDs 106 of second mat 202B. A machine learning model may output first settings for the first subset of dual LEDs 106 and second settings for the second subset of dual LEDs 106, in accordance with the techniques disclosed herein.

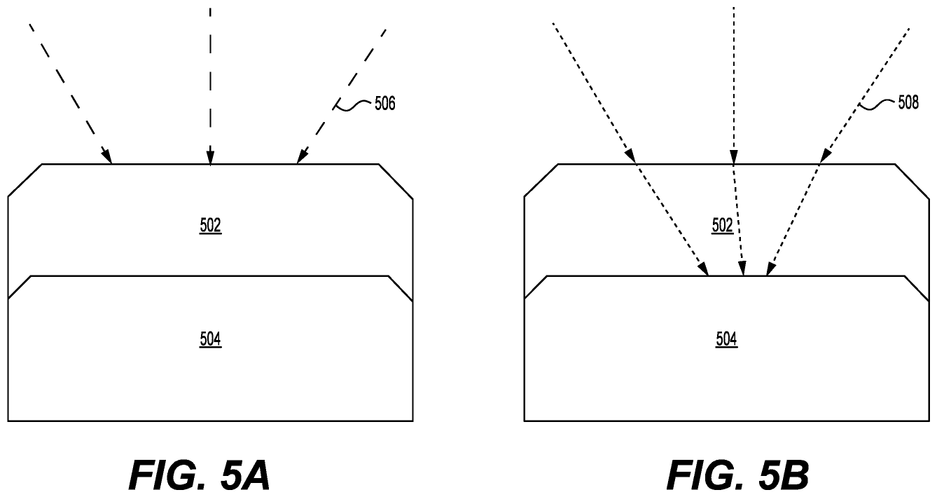

Dual LEDs 106 of mat 102 may be each be configured to emit a first light (e.g., red light) that can interact with cells on a surface as well as a second light (e.g., near infrared light) that can interact with cells deeper than a surface. FIGS. 5A and 5B depict diagrams representing a surface 502 (e.g., a user's skin, hair or head, etc.) and a sub-surface area 504 (e.g., tissue, scalp, muscles, etc.). As disclosed herein, mat 102 may include dual LEDs 106 configured to emit red light, near infrared light, and/or a combination of the two. The ability to emit red light, near infrared light, and/or a combination of the two may result in mat 102 efficiently reducing inflammation, increasing circulation, and/or optimizing functionality of mitochondria. As shown in FIG. 5A, red light 506 may emit from mat 102 and be incident on the surface 502. The red light 506 may interact with the surface 502 to reduce inflammation, increase circulation, and/or optimize functionality of mitochondria, or the like, at the upper surface 502. Additionally, as shown in FIG. 5B, near infrared light 508 may interact with the sub-surface area 504 to reduce inflammation, increase circulation, and/or optimize functionality of mitochondria, or the like, at the sub-surface area 504. Accordingly, using the dual LEDs, mat 102 may have an effect on both the surface 502 and sub-surface area 504 tissue that its light interacts with.

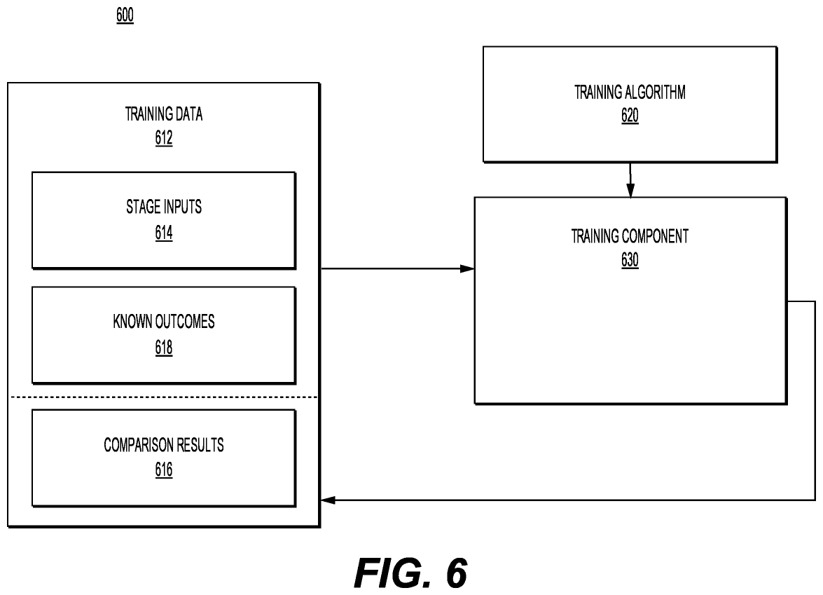

One or more implementations disclosed herein include a machine learning model. For example, as disclosed herein, a machine learning model may output operational parameters or settings to operate mat 102 based on, for example sensor data regarding a health condition. A machine learning model disclosed herein may be trained using the data flow 600 of FIG. 6. As shown in FIG. 6, training data 612 may include one or more of stage inputs 614 and known outcomes 618 related to a machine learning model to be trained. The stage inputs 614 may be from any applicable source including sensor data. The known outcomes 618 may be included for machine learning models generated based on supervised or semi-supervised training. An unsupervised machine learning model may not be trained using known outcomes 618. Known outcomes 618 may include known or desired outputs for future inputs similar to or in the same category as stage inputs 614 that do not have corresponding known outputs.

The training data 612 and a training algorithm 620 may be provided to a training component 630 that may apply the training data 612 to the training algorithm 620 to generate a machine learning model. According to an implementation, the training component 630 may be provided comparison results 616 that compare a previous output of the corresponding machine learning model to apply the previous result to re-train the machine learning model. The comparison results 616 may be used by the training component 630 to update the corresponding machine learning model. The training algorithm 620 may utilize machine learning networks and/or models including, but not limited to a deep learning network such as Deep Neural Networks (DNN), Convolutional Neural Networks (CNN), Fully Convolutional Networks (FCN) and Recurrent Neural Networks (RCN), probabilistic models such as Bayesian Networks and Graphical Models, and/or discriminative models such as Decision Forests and maximum margin methods, large language models (LLM), a generative adversarial network (GAN), a transformer-based model, or a variational autoencoder model, or the like. The machine learning model may be a generative artificial intelligence model which may utilize a large language models (LLM), generative adversarial networks (GAN), transformer-based models, or variational auto encoder models.

In general, any process or operation discussed in this disclosure that is understood to be computer-implementable, such as communicating with an application via a GUI, adjusting mat 102 parameters or settings, etc., may be performed by one or more processors of a computer system. A process or process step performed by one or more processors may also be referred to as an operation. The one or more processors may be configured to perform such processes by having access to instructions (e.g., software or computer-readable code) that, when executed by the one or more processors, cause the one or more processors to perform the processes. The instructions may be stored in a memory of the computer system. A processor may be a central processing unit (CPU), a graphics processing unit (GPU), or any suitable types of processing unit.

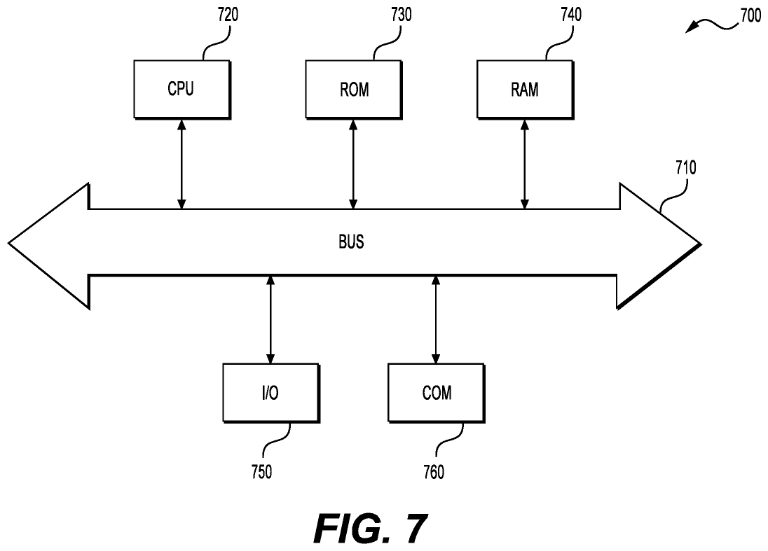

FIG. 7 depicts an example system 700 that may execute techniques presented herein. FIG. 7 is a simplified functional block diagram of a computer that may be configured to execute techniques described herein, according to exemplary embodiments of the present disclosure. Specifically, the computer (or “platform” as it may not be a single physical computer infrastructure) may include a data communication interface 760 for packet data communication. The platform may also include a central processing unit (“CPU”) 720, in the form of one or more processors, for executing program instructions. The platform may include an internal communication bus 710, and the platform may also include a program storage and/or a data storage for various data files to be processed and/or communicated by the platform such as ROM 730 and RAM 740, although the system 700 may receive programming and data via network communications. The system 700 also may include input and output ports 750 to connect with input and output devices such as keyboards, mice, touchscreens, monitors, displays, etc. Of course, the various system functions may be implemented in a distributed fashion on a number of similar platforms, to distribute the processing load. Alternatively, the systems may be implemented by appropriate programming of one computer hardware platform.

The general discussion of this disclosure provides a brief, general description of a suitable computing environment in which the present disclosure may be implemented. In one embodiment, any of the disclosed systems, methods, and/or graphical user interfaces may be executed by or implemented by a computing system consistent with or similar to that depicted and/or explained in this disclosure. Although not required, aspects of the present disclosure are described in the context of computer-executable instructions, such as routines executed by a data processing device, e.g., a server computer, wireless device, and/or personal computer. Those skilled in the relevant art will appreciate that aspects of the present disclosure can be practiced with other communications, data processing, or computer system configurations, including: Internet appliances, hand-held devices (including personal digital assistants (“PDAs”)), wearable computers, all manner of cellular or mobile phones (including Voice over IP (“VoIP”) phones), dumb terminals, media players, gaming devices, virtual reality devices, multi-processor systems, microprocessor-based or programmable consumer electronics, set-top boxes, network PCs, mini-computers, mainframe computers, and the like. Indeed, the terms “computer,” “server,” and the like, are generally used interchangeably herein, and refer to any of the above devices and systems, as well as any data processor.

Aspects of the present disclosure may be embodied in a special purpose computer and/or data processor that is specifically programmed, configured, and/or constructed to perform one or more of the computer-executable instructions explained in detail herein. While aspects of the present disclosure, such as certain functions, are described as being performed exclusively on a single device, the present disclosure may also be practiced in distributed environments where functions or modules are shared among disparate processing devices, which are linked through a communications network, such as a Local Area Network (“LAN”), Wide Area Network (“WAN”), and/or the Internet. Similarly, techniques presented herein as involving multiple devices may be implemented in a single device. In a distributed computing environment, program modules may be located in both local and/or remote memory storage devices.

Aspects of the present disclosure may be stored and/or distributed on non-transitory computer-readable media, including magnetically or optically readable computer discs, hard-wired or preprogrammed chips (e.g., EEPROM semiconductor chips), nanotechnology memory, biological memory, or other data storage media. Alternatively, computer implemented instructions, data structures, screen displays, and other data under aspects of the present disclosure may be distributed over the Internet and/or over other networks (including wireless networks), on a propagated signal on a propagation medium (e.g., an electromagnetic wave(s), a sound wave, etc.) over a period of time, and/or they may be provided on any analog or digital network (packet switched, circuit switched, or other scheme).

Program aspects of the technology may be thought of as “products” or “articles of manufacture” typically in the form of executable code and/or associated data that is carried on or embodied in a type of machine-readable medium. “Storage” type media include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer of the mobile communication network into the computer platform of a server and/or from a server to the mobile device. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to non-transitory, tangible “storage” media, terms such as computer or machine “readable medium” refer to any medium that participates in providing instructions to a processor for execution.

Other embodiments of the disclosure will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A light therapy mat comprising:
a flexible clear portion;
a flexible back portion;
a flexible middle portion between the flexible clear portion and the flexible back portion; and
a plurality of dual light emitting diodes (LEDs) positioned in the flexible middle portion and configured to emit light at a first wavelength and a second wavelength.

2. The light therapy mat of claim 1, wherein the first wavelength is a red light wavelength or a near infrared light wavelength and the second wavelength is the other one of the red light wavelength or the near infrared light wavelength.

3. The light therapy mat of claim 1, wherein the flexible clear portion comprises polyvinylchloride (PVC) material.

4. The light therapy mat of claim 1, wherein the flexible back portion comprises at least one of a nylon, a spandex, or a polyurethane foam composite.

5. The light therapy mat of claim 1, wherein the flexible middle portion comprises a styrene butadiene rubber (SBR) composite.

6. The light therapy mat of claim 1, further comprising a timer configured to activate one or more of the dual LEDs based on at least one of a user input or a machine learning model output.

7. The light therapy mat of claim 1, further comprising an input receptor configured to operate one or more of a timer setting, a power setting, or a dual LED setting.

8. The light therapy mat of claim 7, wherein the input receptor is configured in response to a machine learning output.

9. The light therapy mat of claim 1 further comprising a first mat and a second mat, wherein the first mat and the second mat are connected via a connector.

10. A method for configuring a light therapy mat comprising a plurality of dual light emitting diodes (LEDs), the method comprising:
receiving sensed data from a first sensor associated with the light therapy mat;
providing the sensed data from the first sensor to a machine learning model trained by modifying one or more layers, weighs, synapses, or nodes based on historical or simulated light therapy data;
receiving a first machine learning output from the machine learning model based on the sensed data from the first sensor, the first machine learning output comprising a light therapy mat configuration; and
configuring the light therapy mat based on the light therapy mat configuration.

11. The method of claim 10, wherein the light therapy mat configuration comprises one or more of wavelengths of light, intensities of light, rates of activation, durations of activation, or frequencies associated with the light therapy mat.

12. The method of claim 11, wherein the one or more wavelengths of light are selected from a range of approximately 600 nm-1000 nm.

13. The method of claim 10, wherein the light therapy mat configuration comprises:
a first wavelength or first intensity for application during a first time; and
a second wavelength or second intensity for application during a second time.

14. The method of claim 10, further comprising:
receiving pressure data from a pressure sensor associated with the light therapy mat;
providing the pressure data from the pressure sensor to the machine learning model;
receiving a second machine learning output identifying an active area of the light therapy mat based on the pressure data; and
activating a subset of the plurality of dual LEDs based on the active area.

15. A system for configuring a light therapy mat, the system comprising:
a light therapy mat comprising a plurality of dual light emitting diodes (LEDs) and
a first sensor;
at least one memory storing instructions; and
at least one processor executing the instructions to perform a process, the processor configured to:
receive sensed data from the first sensor associated with the light therapy mat;
provide the sensed data from the first sensor to a machine learning model trained by modifying one or more layers, weighs, synapses, or nodes based on historical or simulated light therapy data;
receive a first machine learning output from the machine learning model based on the sensed data from the first sensor, the first machine learning output comprising a light therapy mat configuration; and
configure the light therapy mat based on the light therapy mat configuration.

16. The system of claim 15, wherein the light therapy mat configuration comprises one or more of wavelengths of light, intensities of light, rates of activation, durations of activation, or frequencies associated with the light therapy mat.

17. The system of claim 16, wherein the one or more wavelengths are selected from a range of approximately 600 nm-1000 nm.

18. The system of claim 15, wherein the light therapy mat configuration comprises:
a first wavelength or first intensity for application during a first time; and
a second wavelength or second intensity for application during a second time.

19. The system of claim 15, wherein the processor is further configured to:
receive pressure data from a pressure sensor associated with the light therapy mat;
provide the pressure data from the pressure sensor to the machine learning model;
receive a second machine learning output identifying an active area of the light therapy mat based on the pressure data; and
activate a subset of the plurality of dual LEDs based on the active area.

20. The system of claim 15, wherein at least one of the at least one processor or the memory is housed within the light therapy mat.
